(19) United States
(12) Patent Application Publication
(10) Pub. No.: US 2025/0378950 A1
(43) Pub. Date: Dec. 11, 2025

(54) VITAL SIGN MONITOR, METHOD FOR OPERATING A VITAL SIGN MONITOR, AND METHOD FOR TRAINING A VITAL SIGN MONITOR

(71) Applicant: ams-OSRAM AG, Premstätten (AT)

(72) Inventors: Ashley Chacon-Alam, Premstätten (AT); Abdelkarim El Qouns, Premstätten (AT); Saumya Saksena, Premstätten (AT); Borys Knysh, Premstätten (AT)

(21) Appl. No.: 18/736,268

(22) Filed: Jun. 6, 2024

(51) Int. Cl. G16H 50/20

(52) U.S. Cl. CPC G16H 50/20

(57) ABSTRACT

In an embodiment a vital sign monitor includes a first sensor for obtaining a time series of a first sensor signal as a first dataset, a second sensor for obtaining a time series of a second sensor signal as a second dataset, a machine-learning based first encoder for extracting a first feature vector from the first dataset, a machine-learning based second encoder for extracting a second feature vector from the first dataset and the second dataset, and a machine-learning based decoder for predicting a vital sign of a person from the first feature vector or the second feature vector.

### TECHNICAL FIELD

The present invention relates to a vital sign motor, to a method for operating a vital sign monitor, and to a method for training a vital sign monitor.

### BACKGROUND

Vital sign monitors for predicting a vital sign of a person are known in the state of the art.

### SUMMARY

Embodiments provide a vital sign monitor. Further embodiments provide a method for operating a vital sign monitor. Yet other embodiments provide a method for training a vital sign monitor.

A vital sign monitor comprises a first sensor for obtaining a time series of a first sensor signal as a first dataset, a second sensor for obtaining a time series of a second sensor signal as a second dataset, a machine-learning based first encoder for extracting a first feature vector from the first dataset, a machine-learning based second encoder for extracting a second feature vector from the first dataset and the second dataset, and a machine-learning based decoder for predicting a vital sign of a person from the first feature vector or the second feature vector.

This vital sign monitor can predict a vital sign of a person from a first dataset obtained using a first sensor or from the first dataset and a second dataset obtained using a second sensor. The usage of the second sensor is thus optional. This allows the vital sign monitor to operate also in a situation when the second sensor or the second sensor signal are not available or not usable. Using both the first dataset and the second dataset may allow to predict the vital sign of the person with an increased precision or reliability.

In a variant of the vital sign monitor, the vital sign is a heart rate or a respiratory rate. These vital signs may provide useful information about the person.

In a variant of the vital sign monitor, the first sensor signal is a bio signal of the person. The first sensor may be an optical sensor, for example.

In a variant of the vital sign monitor, the first dataset is a photoplethysmogram. A photoplethysmogram may contain useful information for predicting a vital sign of a person.

In a variant of the vital sign monitor, the second sensor is an accelerometer. An accelerometer may allow to detect a situation where the vital sign monitor is moved in a way that may also influence the first sensor and the first sensor signal. In this way, the second sensor signal may support the interpretation of the first sensor signal.

In a variant of the vital sign monitor, the first sensor and the second sensor are arranged in a common housing of the vital sign monitor. In this way, the second sensor signal provided by the second sensor may contain information that helps in interpreting the first sensor signal provided by the first sensor.

In a variant of the vital sign monitor, the first encoder or the second encoder comprises a multi-layer perceptron, a convolutional neural network, a recurrent neural network, or an attention-based model. Such encoder architectures have proven to be suitable for extracting a feature vector from a dataset that is formed from a time series of a sensor signal.

In a variant of the vital sign monitor, the first encoder or the second encoder comprises a LeNet or a ResNet architecture. These architectures have proven to be particularly useful for extracting feature vectors from datasets that are composed of a time series of a sensor signal. In a variant of the vital sign monitor, the decoder comprises a neural network with a plurality of fully connected layers. Such a decoder architecture has proven to be useful for predicting a single value from a feature vector.

Some variants of the vital sign monitor further comprise a third sensor for obtaining a time series of a third sensor signal as a third dataset, and a machine-learning based third encoder for extracting a third feature vector from the first dataset and the third dataset. The machine-learning based decoder is adapted for predicting the vital sign of the person from the third feature vector. This variant of the vital sign monitor allows to optionally use also the third sensor and the third sensor signal for predicting the vital sign of the person in the case that the third sensor and the third sensor signal are available. This may allow to predict the vital sign of the person with increased precision or reliability.

Some variants of the vital sign monitor further comprise a third sensor for obtaining a time series of a third sensor signal as a third dataset, and a machine-learning based fourth encoder for extracting a fourth feature vector from the first dataset, the second dataset, and the third dataset. The machine-learning based decoder is adapted for predicting the vital sign of the person from the fourth feature vector. These variants of the vital sign monitor allow to optionally predict the vital sign of the person from the first sensor signal, the second sensor signal and the third sensor signal in the case that all of the first sensor, the second sensor, and the third sensor are available. This may allow to predict the vital sign of the person with a particularly good precision or reliability.

A method for operating a vital sign monitor that is designed as specified above comprises obtaining a time series of a first sensor signal as a first dataset using the first sensor, simultaneously obtaining a time series of a second sensor signal as a second dataset using the second sensor if the second sensor is operational, extracting a feature vector from the first dataset and the second dataset using the second encoder if the second sensor is operational, otherwise extracting the feature vector from the first dataset using the first encoder, and predicting a vital sign of a person from the feature vector using the decoder.

This method allows to predict a vital sign of a person from a first dataset obtained using a first sensor or from the first dataset and a second dataset obtained using a second sensor. The usage of the second sensor is thus optional. This allows the method to be used also in a situation when the second sensor or the second sensor signal are not available or not usable. Using both the first dataset and the second dataset may allow to predict the vital sign of the person with an increased precision or reliability.

Some variants of the method further comprise simultaneously with obtaining the time series of the first sensor signal, obtaining a time series of a third sensor signal as a third dataset using the third sensor if the third sensor is operational, and extracting the feature vector from the first dataset and the third dataset using the third encoder if the third sensor is operational. This may allow to predict the vital sign of the person with an increased precision or reliability in the case that the third sensor is available.

Some variants of the method further comprise simultaneously with obtaining the time series of the first sensor signal, obtaining a time series of a third sensor signal as a third dataset using the third sensor if the third sensor is operational, and extracting the feature vector from the first dataset, the second dataset, and the third dataset using the fourth encoder if the second sensor and the third sensor are operational. In this way, the vital sign of the person is predicted on the basis of the first sensor signal, the second sensor signal and the third sensor signal in the case that all three sensors are available. This may allow for a particularly good precision or reliability of the predicted vital sign.

A method for training a vital sign monitor that is designed as specified above comprises providing a training dataset having a plurality of data records, wherein each data record comprises a time series of a first sensor signal as a first dataset, a time series of a second sensor signal as a second dataset, and a ground truth vital sign. The method further comprises training the first encoder and the decoder using the training dataset in a first training step, wherein for each data record, a first feature vector is extracted from the first dataset using the first encoder, and a predicted vital sign is generated from the first feature vector by the decoder, wherein the training minimizes a difference between the predicted vital sign and the ground truth vital sign in the first training step. The method further comprises calculating a soft label for each data record, wherein for each data record, a first feature vector is extracted from the first dataset using the first encoder, and a predicted vital sign is generated from the first feature vector by the decoder as the soft label. The method further comprises training the second encoder and the decoder using the training dataset in a second training step, wherein for each data record, a first feature vector is extracted from the first dataset using the first encoder, and a first predicted vital sign is generated from the first feature vector by the decoder, a first loss is calculated from a difference between the first predicted vital sign and the soft label, a second feature vector is extracted from the first dataset and the second dataset using the second encoder, and a second predicted vital sign is generated from the second feature vector by the decoder, a second loss is calculated from a difference between the second predicted vital sign and the ground truth vital sign, wherein the training minimizes the first loss and the second loss in the second training step.

This method trains the first encoder and the decoder of the vital sign monitor to predict the vital sign of a person from the first dataset in the first training step. In the second training step, the second encoder and the decoder are trained to predict the vital sign from both the first dataset and the second dataset. The second training step is carried out in a way that the decoder does not lose the ability to predict the vital sign from a first feature vector provided by the first encoder on the basis of only the first dataset. In result, the vital sign monitor is enabled to predict the vital sign only from only the first dataset or optionally from the first dataset and the second dataset.

In a variant of the method, a weighted loss is calculated by weighted addition of the first loss and the second loss for each data record in the second training step. The training minimizes the weighted loss in the second training step. In this way, the second training step trains the decoder to correctly predict the vital sign from both the first feature vector provided by the first encoder and the second feature vector provided by the second encoder.

In a variant of the method, the first encoder is not changed in the second training step. This allows the first encoder to maintain the capabilities that it obtained in the first training step.

### BRIEF DESCRIPTION OF THE DRAWINGS

The above-described properties, features, and advantages of the invention, as well as the way in which they are achieved, will become more clearly and comprehensively understandable in connection with the following description of exemplary variants, which will be explained in more detail in connection with the drawings, in which, in schematic representation:

FIG. 1 shows a vital sign monitor;

FIG. 2 shows a training dataset;

FIG. 3 shows a first training step;

FIG. 4 shows an amended training dataset;

FIG. 5 shows a second training step; and

FIG. 6 shows a further variant of the vital sign monitor.

### DETAILED DESCRIPTION OF ILLUSTRATIVE EMBODIMENTS

FIG. 1 shows a schematic depiction of a vital sign monitor 100. The vital sign monitor 100 is designed for predicting or determining a vital sign of a person who uses the vital sign monitor 100. The vital sign may be a heart rate, a respiratory rate, or a blood pressure, for example. The vital sign monitor 100 may be integrated into a wearable device such as a watch, for example. Alternatively, the vital sign monitor 100 may be integrated into a stationary device, for example.

The vital sign monitor 100 comprises a first sensor 110 for obtaining a time series of a first sensor signal as a first dataset 115. The first sensor signal provided by the first sensor 110 may be a bio signal of the person, for example.

The first dataset 115 formed from a time series of first sensor signals obtained using the first sensor 110 may be a photoplethysmogram, for example. In this case, the first sensor 110 may be an optical sensor comprising one or more light emitters and one or more light detectors, for example.

Alternatively, the first dataset 115 may be an electrocardiogram, for example. In this case, the first sensor 110 may include one or more electrodes, for example.

The first dataset 115 may contain a few hundred data points, for example. In one variant, the first dataset 115 comprises 256 data points. Each data point of the first dataset 115 may be a floating point number, for example. The time series of the first sensor signal that forms the first dataset 115 may be recorded at 100 Hz, for example.

The vital sign monitor 100 further comprises a second sensor 120 for obtaining a time series of a second sensor signal as a second dataset 125. The second sensor 120 may be an accelerometer, for example. In this case, the second dataset 125 is formed by a time series of accelerometer signals measured using the second sensor 120.

The vital sign monitor 100 is designed to obtain the first dataset 115 and the second dataset 125 simultaneously. In this way, the first dataset 115 and the second dataset 125 are recorded under the same conditions. It is useful if the first sensor 110 and the second sensor 120 are arranged in a common housing 105 of the vital sign monitor 100. In this way, the second dataset 125 may provide context for interpreting the first dataset 115 and vice versa. As an example, in case that the second sensor 120 is an accelerometer and that the first sensor 110 and the second sensor 120 are arranged in den common housing 105, one may assume that the first sensor 110 experiences a similar or an identical acceleration while recording the first dataset 115 as the second sensor 120.

The second sensor 120 may record the second dataset 125 at the same sampling rate as the recording of the first dataset 115, or at a lower or higher sampling rate. The time series of the second sensor signal may be recorded as the second dataset 125 at 100 Hz, for example.

The vital sign monitor 100 comprises a machine-learning based first encoder 210 for extracting a first feature vector 215 from the first dataset 115. The vital sign monitor 100 further comprises a machine-learning based second encoder 220 for extracting a second feature vector 225 from the first dataset 115 and the second dataset 125. The first feature vector 215 and the second feature vector 225 are vectors in a common feature space. In most variants, the first feature vector 215 and the second feature vector 225 comprise the same dimension (number of elements). It is convenient if the dimension of the first feature vector 215 and the second feature vector 225 is smaller than the dimension of the first dataset 115 and the dimension of the second dataset 125. The first feature vector 215 and the second feature vector 225 may comprise a size of 100 floating point values, for example.

The first feature vector 215 and the second feature vector 225 contain significant features extracted from the first dataset 115 and the second dataset 125. To be able to extract these features, the first encoder 210 and the second encoder 220 each comprise a machine-learning based architecture and have been trained as explained below. Each of the first encoder 210 and the second encoder 220 may comprise a multi-layer perceptron, a convolutional neural network, a recurrent neural network, or an attention-based model, for example. Each of the first encoder 210 and the second encoder 220 may comprise a LeNet or a ResNet architecture, for example.

The vital sign monitor 100 further comprises a machine-learning based decoder 300 for predicting a vital sign 305 of the person using the vital sign monitor 100 from the first feature vector 215 or from the second feature vector 225. The decoder 300 comprises a machine-learning based architecture and has been trained as explained below. The decoder 300 may comprise a neuronal network with a plurality of fully-connected layers, for example. The decoder 300 may comprise the architecture of a regressor, for example.

The second sensor 120 and the second sensor signal provided by the second sensor 120 may not be available at all times during operation of the vital sign monitor 100. This may be due to circumstances that prevent operation of the second sensor 120 or that prevent the second sensor 120 from providing sensible second sensor data. In some variants of the vital sign monitor 100, it may possible to switch off the second sensor 120 to conserve energy, for example.

The vital sign monitor 100 is designed to operate and be able to predict the vital sign 305 of the person using the vital sign monitor 100 both in situations when the second sensor 120 is operational and is not operational. In the case that the second sensor 120 and the second sensor signal are not available, only the first dataset 115 is obtained using the first sensor 110. The first encoder 210 is used for extracting the first feature vector 215 from the first dataset 115. The decoder 300 predicts the vital sign 305 from the first feature vector 215. In this mode of operating the vital sign monitor 100, the second encoder 220 is not used.

In case that the second sensor 120 and the second sensor signal are available, the first dataset 115 is obtained using the first sensor 110. Simultaneously, the second dataset 125 is obtained using the second sensor 120. The second encoder 220 is used for extracting the second feature vector 225 from the first dataset 115 and the second dataset 125. The decoder 300 is used for predicting the vital sign 305 of the person from the second feature vector 225. In this mode of operating the vital sign monitor 100, the first encoder 210 is not used.

Using the second encoder 220 for extracting the second feature vector 225 from the first dataset 115 and the second dataset 125, and predicting the vital sign 305 from the second feature vector 225 using the decoder 300 may allow to predict the vital sign 305 with increased precision or reliability, because the second dataset 125 may provide additional context for the interpretation of the first dataset 115. In the case that the second sensor 120 is an accelerometer, for example, the second dataset 125 may help to compensate motion-based noise and artifacts in the first dataset 115.

In some variants of the vital sign monitor 100, an alternative mode of operation can be used in the case that the second sensor 120 and the second sensor signal are available. In this mode of operation, the first encoder 210 is used to extract the first feature vector 215 from the first dataset 115. At the same time, the second encoder 220 is used for extracting the second feature vector 225 from the first dataset 115 and the second dataset 125. After extracting the first feature vector 215 and the second feature vector 225, either the first feature vector 215 or the second feature vector 225 is chosen for predicting the vital sign 305 using the decoder 300. The selection of the first feature vector 215 or the second feature vector 225 may be based on an evaluation of the quality of the first feature vector 215 and the quality of the second feature vector 225 using a pre-defined quality criterion, for example.

The vital sign monitor 100 may be designed to operate continuously over a long period of time. In this case, the first sensor data provided by the first sensor 110 and the optional second sensor data provided by the second sensor 120 are divided into consecutive segments of equal length that each form first datasets 115 and second datasets 125. Each first dataset 115, optionally paired with a second dataset 125, is used for one prediction of the vital sign 305. Consecutive first datasets 115 and second datasets 125 are used for consecutive predictions of the vital sign 305, allowing for a determination of a temporal trend of the vital sign 305.

In the following, a method for training the vital sign monitor 100 will be explained. Training the vital sign monitor 100 includes training the machine-learning based first encoder 210, the machine-learning based second encoder 220 and the machine-learning based decoder 300.

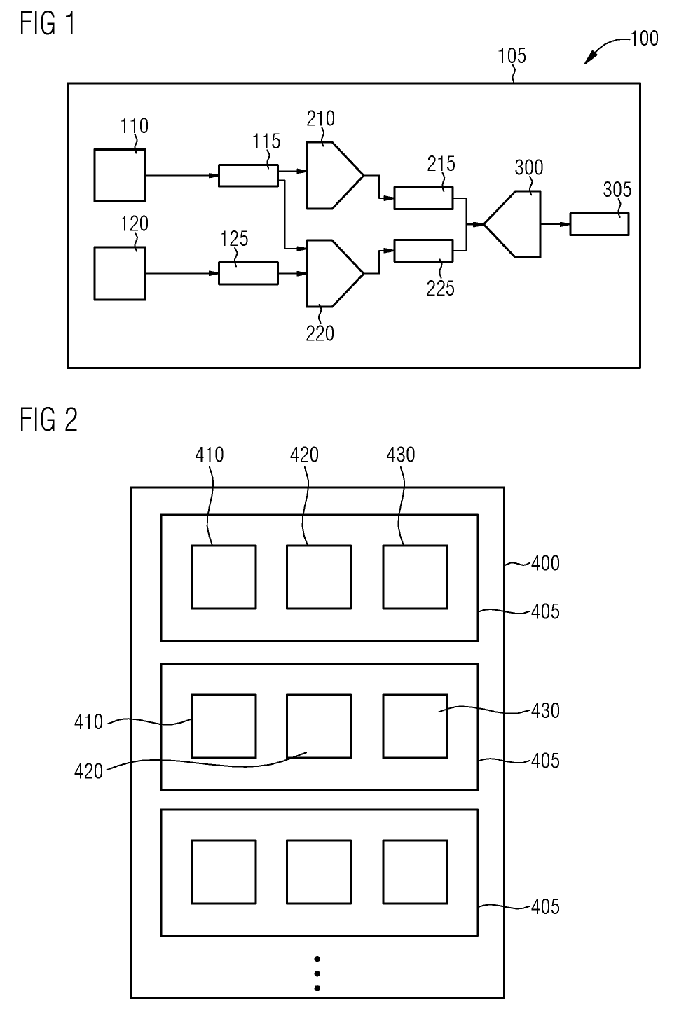

The method starts with providing a training dataset 400 that is schematically depicted in FIG. 2. The training dataset 400 comprises a plurality of data records 405. Each data record 405 comprises a time series of a first sensor signal as a first dataset 410, a time series of a second sensor signal as a second dataset 420, and a ground truth vital sign 430 of a person.

The first datasets 410 of the data records 405 are similar to the first dataset 115 that can be obtained with the first sensor 110 of the vital sign monitor 100. The second datasets 420 of the data records 405 are similar to the second dataset 125 that can be obtained using the second sensor 120 of the vital sign monitor 100. The first datasets 410 and the second datasets 420 of the data records 405 can be generated by performing measurements on an real person using sensors similar to the first sensor 110 and the second sensor 120, for example. The ground truth vital sign 430 of each data record 405 is a vital sign of the person that may be determined using another measurement device simultaneously with recording the first dataset 410 and the second dataset 420 of the corresponding data record 405.

Alternatively, the first datasets 410, the second datasets 420, and the ground truth vital signs 430 of the data records 405 of the training dataset 400 may be created synthetically.

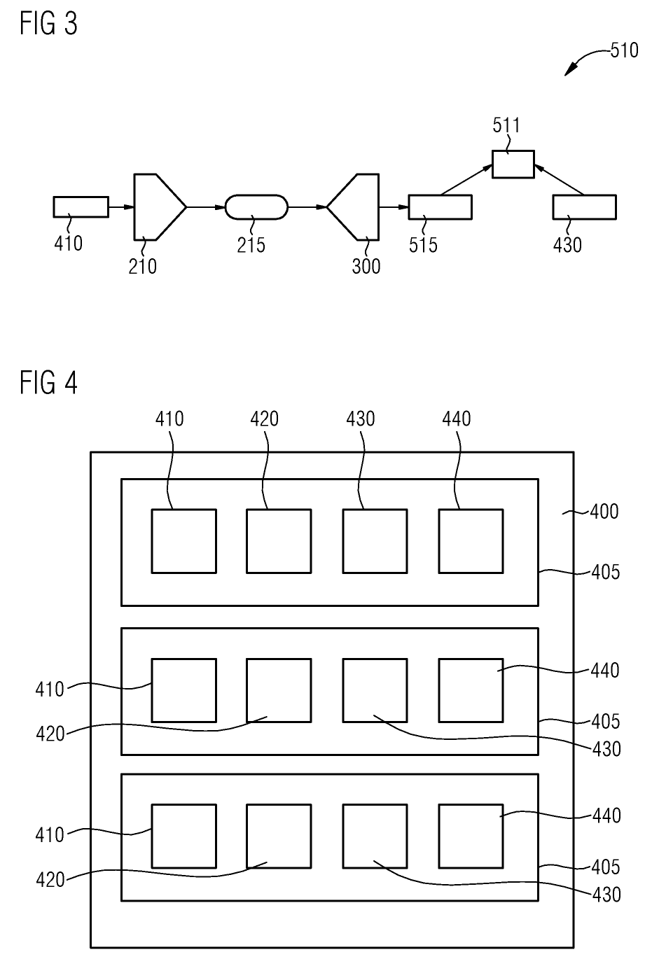

FIG. 3 schematically depicts a first training step 510 that trains the first encoder 210 and the decoder 300 using the training dataset 400. In the first training step 510, the following steps are carried out for each data record 405 of the training dataset 400.

A first feature vector 215 is extracted from the first dataset 410 of the respective data record 405 using the first encoder 210. A predicted vital sign 515 is generated from the first feature vector 215 by the decoder 300. A loss 511 is calculated on the basis of a difference between the predicted vital sign 515 and the ground truth vital sign 430 of the respective data record 405. Then the first encoder 210 and the decoder 300 are adapted in dependence of the loss 511.

In this way, the first training step 510 serves to minimize the differences between the predicted vital signs 515 and the ground truth vital signs 430 for each data record 405 of the training dataset 400.

After completion of the first training step 510, a soft label 440 is calculated for each data record 405 of the training dataset 400 using the optimal configuration of the first encoder 210 and the decoder 300 that has been found in the first training step 510. The training dataset 400 with the added soft labels 440 is schematically depicted in FIG. 4.

To calculate the soft labels 440, for each data record 405, a first feature vector 215 is extracted from the first dataset 410 using the optimal configuration of the first encoder 210. A predicted vital sign is generated from the first feature vector 215 using the optimal configuration of the decoder 300. The predicted vital sign is used as the soft label 440.

Afterwards, a second training step 520 is carried out that is schematically depicted in FIG. 5. The second training step 520 serves to train the second encoder 220 and the decoder 300 while maintaining the capabilities that the decoder 300 has gained in the first training step 510 as good as possible. In most variants of the training method, the first encoder 210 is not changed anymore in the second training step 520.

In the second training step 520, the following steps are carried out for each data record 405 of the amended training dataset 400 depicted in FIG. 4.

A first feature vector 215 is extracted from the first dataset 410 of the respective data record 405 using the first encoder 210. A first predicted vital sign 525 is generated from the first feature vector 215 by the decoder 300. A first loss 521 is calculated on the basis of a difference between the first predicted vital sign 525 and the soft label 440 of the respective data record 405. The first loss 521 may also be referred to as a knowledge distillation loss.

A second feature vector 225 is extracted from the first dataset 410 and the second dataset 420 of the respective data record 405 using the second encoder 220. A second predicted vital sign 526 is generated from the second feature vector 225 by the decoder 300. A second loss 522 is calculated from a difference between the second predicted vital sign 526 and the ground truth vital sign 430.

Then the second encoder 220 and the decoder 300 are modified on the basis of the first loss 521 and the second loss 522 such that the first loss 521 and the second loss 522 are minimized. To this end, a weighted loss 523 may be calculated by weighted addition of the first loss 521 and the second loss 522, for example. In this example, the second encoder 220 and the decoder 300 are adjusted on the basis of the weighted loss 523 such that the weighted loss 523 is minimized.

After the second training step 520, the training of the first encoder 210, the second encoder 220 and the decoder 300 of the vital sign monitor 100 is completed.

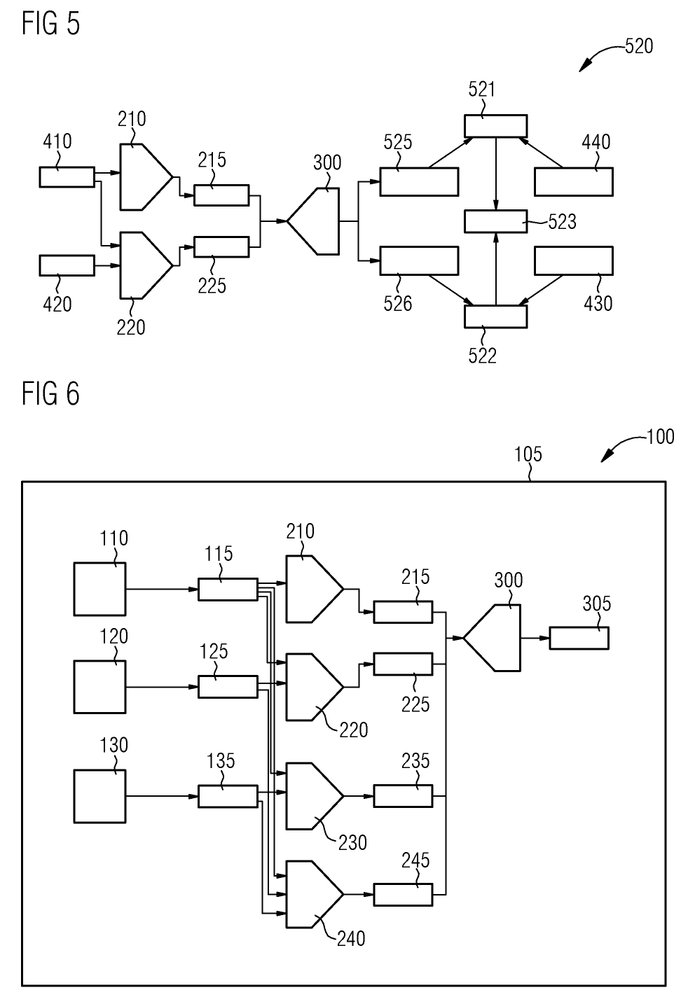

FIG. 6 shows a schematic depiction of a further variant of the vital sign monitor 100. This variant of the vital sign monitor 100 comprises all components described in conjunction with FIG. 1. Additionally, this variant of the vital sign monitor 100 comprises a third sensor 130 for obtaining a time series of a third sensor signal as a third dataset 135. This variant of the vital sign monitor 100 further comprises a machine-learning based third encoder 230 for extracting a third feature vector 235 from the first dataset 115 and the third dataset 135. This variant of the vital sign monitor 100 further comprises a machine-learning based fourth encoder 240 for extracting a fourth feature vector 245 from the first dataset 115, the second dataset 125, and the third dataset 135. In this variant of the vital sign monitor 100, the decoder 300 is adapted for predicting the vital sign 305 of the person from the first feature vector 215, the second feature vector 225, the third feature vector 235, or the fourth feature vector 245.

In operation of this variant of the vital sign monitor 100, a situation may arise in which the first sensor 110 and the third sensor 130 are operational but the second sensor 120 is not available. In this case, operating the vital sign monitor 100 comprises obtaining a time series of the first sensor signal as the first dataset 115 using the first sensor 110, and simultaneously obtaining a time series of the third sensor signal as the third dataset 135 using the third sensor 130. Then the third feature vector 235 is extracted from the first dataset 115 and the third dataset 135 using the third encoder 230. The vital sign 305 is predicted from the third feature vector 235 using the decoder 300.

During operation of the vital sign monitor 100 depicted in FIG. 6, another situation may arise where each of the first sensor 110, the second sensor 120 and the third sensor 130 is available and operational. In this case, operating the vital sign monitor 100 may comprise obtaining a time series of the first sensor signal as the first dataset 115 using the first sensor 110, and simultaneously obtaining a time series of the second sensor signal as the second dataset 125 using the second sensor 120, and simultaneously obtaining a time series of the third sensor signal as the third dataset 135 using the third sensor 130. Then, the fourth feature vector 245 is extracted from the first dataset 115, the second dataset 125, and the third dataset 135 using the fourth encoder 240. The vital sign 305 is predicted from the fourth feature vector 245 using the decoder 300.

Training the variant of the vital sign monitor 100 depicted in FIG. 6 requires a training dataset 400 where each data record 405 includes a third dataset in addition to the components shown in FIGS. 2 and 4. These third datasets mimic the third dataset 135 that can be obtained as a time series of third sensor signals using the third sensor 130.

In training the variant of the vital sign monitor 100 depicted in FIG. 6, second soft labels are calculated after the second training step 520 using the second encoder 220 and the decoder 300. This is followed by a third training step that trains the third encoder 230 and the decoder 300 but leaves the first encoder 210 and the second encoder 220 unmodified. In the third training step, a first loss is again calculated from the difference between the first predicted vital sign 525 predicted by the first encoder 210 and the decoder 300, and the soft label 440. A second loss is calculated on the basis of a difference between the second predicted vital sign 526 predicted by the second encoder 220 and the decoder 300, and the second soft label. A third loss is calculated from a difference between a vital sign predicted by the third encoder 230 and the decoder 300, and the ground truth vital sign 430 of the respective data record 405. The third encoder 230 and the decoder 300 are modified to minimize the three losses.

After the third training step, further soft labels are calculated using the third encoder 230 and the decoder 300 and the fourth encoder 240 is trained in a similar manner in a fourth training step.

Further variants of the vital sign monitor 100 comprise only the third encoder 230 or only the fourth encoder 240. Other variants of the vital sign monitor 100 comprise even further sensors and encoders.

The invention has been illustrated and described in more detail with the aid of exemplary variants. The invention is not, however, restricted to the examples disclosed. Rather, other variations may be derived therefrom by the person skilled in the art.

What is claimed is:

1. A vital sign monitor comprising:
a first sensor configured to obtain a time series of a first sensor signal as a first dataset;
a second sensor configured to obtain a time series of a second sensor signal as a second dataset;
a machine-learning based first encoder configured to extract a first feature vector from the first dataset;
a machine-learning based second encoder configured to extract a second feature vector from the first dataset and the second dataset; and
a machine-learning based decoder configured to predict a vital sign of a person from the first feature vector or the second feature vector.

2. The vital sign monitor according to claim 1, wherein the vital sign is a heart rate or a respiratory rate.

3. The vital sign monitor according to claim 1, wherein the first sensor signal is a bio signal of the person.

4. The vital sign monitor according to claim 1, wherein the first dataset is a photoplethysmogram.

5. The vital sign monitor according to claim 1, wherein the second sensor is an accelerometer.

6. The vital sign monitor according to claim 1, wherein the first sensor and the second sensor are arranged in a common housing of the vital sign monitor.

7. The vital sign monitor according to claim 1, wherein the first encoder or the second encoder comprises a multi-layer perceptron, a convolutional neural network, a recurrent neural network, or an attention-based model.

8. The vital sign monitor according to claim 1, wherein the first encoder or the second encoder comprises a LeNet or a ResNet architecture.

9. The vital sign monitor according to claim 1, wherein the decoder comprises a neural network with a plurality of fully-connected layers.

10. The vital sign monitor according to claim 1, further comprising:
a third sensor configured to obtain a time series of a third sensor signal as a third dataset; and
a machine-learning based third encoder configured to extract a third feature vector from the first dataset and the third dataset ,
wherein the machine-learning based decoder is configured to predict the vital sign of the person from the third feature vector.

11. The vital sign monitor according to claim 1, further comprising:
a third sensor configured to obtain a time series of a third sensor signal as a third dataset; and
a machine-learning based fourth encoder configured to extract a fourth feature vector from the first dataset, the second dataset, and the third dataset ,
wherein the machine-learning based decoder is configured to predict the vital sign of the person from the fourth feature vector.

12. A method for operating a vital sign monitor, wherein the vital sign monitor comprises a first sensor, a second sensor, a machine-learning based first encoder, a machine-learning based second encoder, and a machine-learning based decoder, the method comprising:
obtaining a time series of a first sensor signal as a first dataset using the first sensor;
simultaneously obtaining a time series of a second sensor signal as a second dataset using the second sensor when the second sensor is operational;
extracting a feature vector from the first dataset and the second dataset using the second encoder when the second sensor is operational, otherwise extracting the feature vector from the first dataset using the first encoder; and
predicting a vital sign of a person from the feature vector using the decoder.

13. The method according to claim 12, further comprising:
simultaneously with obtaining the time series of the first sensor signal, obtaining a time series of a third sensor signal as a third dataset using a third sensor of the vital sign monitor when the third sensor is operational; and
extracting the feature vector from the first dataset and the third dataset using a third encoder if the third sensor is operational.

14. The method according to claim 12, further comprising:
simultaneously with obtaining the time series of the first sensor signal, obtaining a time series of a third sensor signal as a third dataset using a third sensor of the vital sign monitor when the third sensor is operational; and
extracting the feature vector from the first dataset, the second dataset, and the third dataset using a fourth encoder if the second sensor and the third sensor are operational.

15. A method for training a vital sign monitor, wherein the vital sign monitor comprises a first sensor, a second sensor, a machine-learning based first encoder, a machine-learning based second encoder, and a machine-learning based decoder, the method comprising:
providing a training dataset having a plurality of data records, wherein each data record comprises a time series of a first sensor signal as a first dataset, a time series of a second sensor signal as a second dataset, and a ground truth vital sign;
training the first encoder and the decoder using the training dataset in a first training step,
wherein, for each data record, a first feature vector is extracted from the first dataset using the first encoder, and a predicted vital sign is generated from the first feature vector by the decoder, and
wherein training minimizes a difference between the predicted vital sign and the ground truth vital sign in the first training step;
calculating a soft label for each data record, wherein, for each data record, the first feature vector is extracted from the first dataset using the first encoder, and the predicted vital sign is generated from the first feature vector by the decoder as the soft label; and
training the second encoder and the decoder using the training dataset in a second training step,
wherein, for each data record,
the first feature vector is extracted from the first dataset using the first encoder and a first predicted vital sign is generated from the first feature vector by the decoder,
a first loss is calculated from a difference between the first predicted vital sign and the soft label,
a second feature vector is extracted from the first dataset and the second dataset using the second encoder and a second predicted vital sign is generated from the second feature vector by the decoder, and
a second loss is calculated from a difference between the second predicted vital sign and the ground truth vital sign, and
wherein the training minimizes the first loss and the second loss in the second training step.

16. The method according to claim 15,
wherein a weighted loss is calculated by weighted addition of the first loss and the
second loss for each data record in the second training step,
wherein the training minimizes the weighted loss in the second training step.

17. The method according to claim 15, wherein the first encoder is not changed in the second training step.
